(19) United States
(12) Patent Application Publication
(10) Pub. No.: US 2023/0295224 A1
(43) Pub. Date: Sep. 21, 2023

(54) METHODS OF PROCESSING A FLUID

(71) Applicant: Genzyme Corporation, Cambridge, MA (US)

(72) Inventors: Michael Coolbaugh, Waltham, MA (US); Shashi Malladi, Westborough, MA (US)

(21) Appl. No.: 18/174,575

(22) Filed: Feb. 24, 2023

Related U.S. Application Data
(60) Provisional application No. 63/322,133, filed on Mar. 21, 2022.

(51) Int. Cl. C07K 1/36; B01D 61/14

(52) U.S. Cl. CPC C07K 1/36; B01D 61/145; B01D 2315/10

(57) ABSTRACT

Provided herein are methods of processing a fluid comprising a recombinant therapeutic protein that include the use of a bioprocessing system comprising integrated unit operations, including cell removal, cell retention, chromatography, virus inactivation, virus filtration, TFF, and formulation. Also provided are bioprocessing systems that include these unit operations.

### CROSS-REFERENCE TO RELATED APPLICATIONS

This application claims priority to U.S. Provisional Patent Application Serial No. 63/322,133, filed Mar. 21, 2022; the entire contents of which are herein incorporated by reference.

### TECHNICAL FIELD

This invention relates to methods of biotechnology and the biomanufacturing of recombinant therapeutic proteins.

### BACKGROUND

Mammalian cells containing a nucleic acid that encodes a recombinant therapeutic protein are often used to produce therapeutically or commercially important proteins. In the current environment of diverse product pipelines, biotechnology companies are increasingly driven to develop innovative solutions for highly flexible and cost-effective manufacturing of therapeutic protein drug substances.

### SUMMARY

The present invention is based, at least in part, on the discovery that the combined application of a back pressure regulator (BPR) and a booster pump (e.g., a centrifugal pump) to a tangential flow filtration (TFF) unit operation can achieve and maintain the targeted retentate concentration and maintain the flow rates of retentate and permeate in ultrafiltration (UF) and diafiltration (DF) operations.

Provided herein are methods of processing a fluid including a recombinant therapeutic protein that include: (a) providing a bioprocessing system including: (i) a unit operation, (ii) at least one inlet on the unit operation, (iii) at least one outlet on the unit operation, (iv) an inlet conduit in fluid communication with at least one inlet of the unit operation, (v) an outlet conduit in fluid communication with at least one outlet of the unit operation, (vi) a back pressure regulator disposed in the outlet conduit, and (vii) a booster pump disposed in the outlet conduit that follows downstream of the back pressure regulator; (b) flowing the fluid including the recombinant therapeutic protein through the inlet conduit and into the at least one inlet of the unit operation, processing the fluid within the unit operation to obtain a processed fluid, and then flowing a portion of the processed fluid into the outlet conduit; and (c) maintaining a target flow rate and/or pressure through the outlet conduit using the back pressure regulator and the booster pump.

In some embodiments, the unit operation is selected from the group consisting of: a cell removal unit operation, a cell retention unit operation, a tangential flow filtration unit operation, a chromatography unit operation, a viral inactivation unit operation, a virus removal filtration unit operation and a formulation unit operation.

Also provided herein are methods of processing a fluid including a recombinant therapeutic protein that include: (a) providing a bioprocessing system including: (i) a tangential flow filtration (TFF) unit with a permeate side and a retentate side, (ii) at least one inlet on the TFF unit, (iii) at least two outlets on the TFF unit, where one of the at least two outlets is on the permeate side of the TFF unit and one of the at least two outlets is on the retentate side of the TFF unit, (iv) an inlet conduit in fluid communication with at least one inlet of the TFF unit, (v) a permeate conduit in fluid communication with the outlet on the permeate side of the TFF unit, (vi) a retentate conduit in fluid communication with the outlet on the retentate side of the TFF unit, (vii) a back pressure regulator disposed in the permeate conduit or the retentate conduit, and (viii) a booster pump disposed in the permeate conduit or the retentate conduit that follows downstream of the back pressure regulator; (b) flowing the fluid including the recombinant therapeutic protein through the inlet conduit and into the at least one inlet of the TFF unit, and separating the fluid into retentate and permeate streams based on the pore size or molecular weight cutoff of the TFF unit, and then flowing those permeate and retentate streams into the permeate and retentate conduits, respectively; and (c) maintaining a target flow rate and/or pressure through the permeate or retentate conduit using the back pressure regulator and the booster pump.

In some embodiments, the TFF unit has a molecular weight cutoff from about 1 kDa to about 1000 kDa. In some embodiments, the TFF unit has a molecular weight cutoff from about kDa to about 100 kDa.

In some embodiments, the TFF unit has an average pore size from about 10 nm to about 100 nm. In some embodiments, the TFF unit has an average pore size from about 15 nm to about 35 nm. In some embodiments, the TFF unit has an average pore size from about 0.1 μm to about 10 μm.

In some embodiments, the recombinant therapeutic protein in the fluid in the inlet conduit (also referred as feed stream) has a concentration from about 0.1 g/L to about 300 g/L. In some embodiments, the recombinant therapeutic protein in the feed stream in the inlet conduit has a concentration from about 0.1 g/L to about 150 g/L.

In some embodiments, the recombinant therapeutic protein in the retentate stream has a concentration from about 0.1 g/L to about 300 g/L.

In some embodiments, the recombinant therapeutic protein in the retentate stream has a concentration from about 5 g/L to about 225 g/L.

In some embodiments, the recombinant therapeutic protein in the permeate stream has a concentration from about 0 g/L to about 100 g/L. In some embodiments, the recombinant therapeutic protein in the permeate stream has a concentration from about 0.1 g/L to about 25 g/L.

In some embodiments, the TFF unit is included of one or more filters each with filter areas of about 50 cm² to about 10 m². In some embodiments, the TFF unit is included of one or more filters each with filter areas of about 0.1 m² to about 5 m².

In some embodiments, the booster pump is a dynamic pump. In some embodiments, the dynamic pump is a centrifugal pump.

In some embodiments, step (b) includes continuously flowing the fluid at an average rate from about 0.1 mL/minute to about 10 L/minute.

In some embodiments, step (b) includes continuously flowing the fluid at an average rate from about 1 mL/minute to about 1 L/minute.

In some embodiments, the bioprocessing system further includes one or more sensor(s).

In some embodiments, at least one of the one or more sensor(s) is disposed in the inlet conduit. In some embodiments, at least one of the one or more sensor(s) is disposed in the permeate conduit.

In some embodiments, at least one of the one or more sensor(s) is disposed in the retentate conduit.

In some embodiments, the recombinant therapeutic protein concentration is measured using the one or more sensor(s) that has a sampling time of less than about 2 minutes.

In some embodiments, the recombinant therapeutic protein concentration is measured using UV absorbance, refractive index, Fourier Transform Infrared Spectroscopy (FTIR), or Raman spectroscopy.

In some embodiments, the recombinant therapeutic protein concentration is measured using UV absorbance, and wherein the UV absorbance is measured between about 200 nm to about 400 nm.

In some embodiments, the recombinant therapeutic protein concentration is measured using UV absorbance, and wherein the UV absorbance is measured between about 260 nm to about 320 nm.

In some embodiments, the UV absorbance is measured with a flowcell with a fixed pathlength.

In some embodiments, the UV absorbance is measured with a flowcell with a variable pathlength.

In some embodiments, the method further includes, prior to step (a), performing one or more second unit operation(s) on the recombinant therapeutic protein, wherein the one or more second unit operation(s) is/are integrated upstream of the bioprocessing system, such that one of the one or more second unit operation is in fluid communication with the inlet conduit of the bioprocessing system.

In some embodiments, the one or more second unit operation(s) is/are selected from the group consisting of: a bioreactor unit operation, a cell removal unit operation, a cell retention unit operation, a chromatography unit operation, a viral inactivation unit operation, a virus removal unit operation, an ultrafiltration unit operation, and a diafiltration unit operation.

In some embodiments, the method further includes, after step (c), performing one or more second unit operation(s), wherein the one or more second unit operation(s) is/are integrated downstream of the bioprocessing system, such that one of the one or more second unit operation(s) is in fluid communication with the permeate conduit of the bioprocessing system.

In some embodiments of any of the methods described herein, the method further includes, after step (c), performing one or more second unit operation(s), wherein the one or more second unit operation(s) is/are integrated downstream of the bioprocessing system, such that one of the one or more second unit operation(s) is in fluid communication with the retentate conduit of the bioprocessing system.

In some embodiments, the one or more second unit operation(s) is/are selected from the group consisting of: a chromatography unit operation, a viral inactivation unit operation, a virus removal unit operation, an ultrafiltration unit operation, a diafiltration unit operation, or a formulation unit operation.

Provided herein are bioprocessing systems including: (i) a unit operation; (ii) at least one inlet on the unit operation; (iii) at least one outlet on the unit operation; (iv) an inlet conduit in fluid communication with at least one inlet of the unit operation; (v) an outlet conduit in fluid communication with at least one outlet of the unit operation; (vi) a back pressure regulator disposed in the outlet conduit, and (vii) a booster pump disposed in the outlet conduit that follows downstream of the back pressure regulator, wherein: the bioprocessing system is configured to flow fluid including the recombinant therapeutic protein through the inlet conduit and into the at least one inlet of the unit operation, processing the fluid within the unit operation to obtain a processed fluid, and then flowing a portion of the processed fluid into the outlet conduit and the back pressure regulator and the booster pump are configured to maintain a target flow rate and/or pressure through the outlet conduit.

In some embodiments, the unit operation is selected from the group consisting of a cell removal unit operation, a cell retention unit operation, a tangential flow filtration unit operation, a chromatography unit operation, a viral inactivation unit operation, a virus removal unit operation, and a formulation unit operation.

Also provided herein are bioprocessing systems that include: (i) a tangential flow filtration (TFF) unit having at least one inlet and at least two outlets, wherein one of the at least two outlets is on the permeate side of the TFF unit and one of the at least two outlets is on the retentate side of the TFF unit; (ii) an inlet conduit in fluid communication with the at least one inlet of the TFF unit; (iii) a permeate conduit in fluid communication with the outlet on the permeate side of the TFF unit; (iv) a retentate conduit in fluid communication with the outlet on the retentate side of the TFF unit; (v) a back pressure regulator disposed in the permeate or retentate conduit, and (vi) a booster pump disposed in the permeate or retentate conduit that follows downstream of the back pressure regulator, wherein: the bioprocessing system is configured to flow fluid including a recombinant therapeutic protein through the inlet conduit and into the at least one inlet of the TFF unit, and separate the fluid into retentate and permeate streams based on the pore size or molecular weight cutoff of the TFF unit, and then flow those permeate and retentate streams into the permeate and retentate conduits; and the back pressure regulator and the booster pump are configured to maintain a target flow rate and/or pressure through the permeate or retentate conduit.

In some embodiments, the TFF unit has a molecular weight cutoff from about 1 kDa to about 1000 kDa. In some embodiments, the TFF unit has a molecular weight cutoff from about kDa to about 100 kDa. In some embodiments, the TFF unit has an average pore size from about 10 nm to about 100 nm. In some embodiments, the TFF unit has an average pore size from about 15 nm to about 35 nm. In some embodiments, the TFF unit has an average pore size from about 0.1 μm to about 10 μm.

In some embodiments of any of the bioprocessing systems described herein, the recombinant therapeutic protein in the fluid in the inlet conduit has a concentration from about 0.1 g/L to about 300 g/L.

In some embodiments, the recombinant therapeutic protein in the fluid in the inlet conduit has a concentration from about 0.1 g/L to about 150 g/L.

In some embodiments, of any of the bioprocessing system described herein, the recombinant therapeutic protein in the retentate stream has a concentration from about 0.1 g/L to about 300 g/L. In some embodiments, the recombinant therapeutic protein in the retentate stream has a concentration from about 5 g/L to about 225 g/L.

In some embodiments of any of the bioprocessing systems described herein, the recombinant therapeutic protein in the permeate stream has a concentration from about 0 g/L to about 100 g/L.

In some embodiments, the recombinant therapeutic protein in the permeate stream has a concentration from about 0.1 g/L to about 25 g/L.

In some embodiments of any of the bioprocessing systems described herein, the TFF unit is included of one or more filters each with filter areas of about 50 cm² to about 10 m².

In some embodiments, the TFF unit is included of one or more filters each with filter areas of about 0.1 m² to about 5 m².

In some embodiments of any of the bioprocessing systems described herein, the booster pump is a dynamic pump. In some embodiments, the dynamic pump is a centrifugal pump.

In some embodiments of any of the bioprocessing systems described herein, the bioprocessing system further includes one or more sensor(s). In some embodiments, at least one of the one or more sensor(s) is disposed in the inlet conduit.

In some embodiments of any of the bioprocessing systems described herein, at least one of the one or more sensor(s) is disposed in the permeate conduit.

In some embodiments of any of the bioprocessing systems described herein, at least one of the one or more sensor(s) is disposed in the retentate conduit.

In some embodiments of any of the bioprocessing systems described herein, the one or more sensors are configured to measure the recombinant therapeutic protein concentration with a sampling time of less than about 2 minutes.

In some embodiments of any of the bioprocessing systems described herein, the one or more sensors are configured to measure the recombinant therapeutic protein concentration using UV absorbance, refractive index, Fourier Transform Infrared Spectroscopy (FTIR), or Raman spectroscopy.

In some embodiments, the one or more sensors are configured to measure the recombinant therapeutic protein concentration using UV absorbance between about 200 nm to about 400 nm.

In some embodiments, the one or more sensors are configured to measure the recombinant therapeutic protein concentration using UV absorbance between about 260 nm to about 320 nm.

In some embodiments of any of the bioprocessing systems described herein, at least one of one of the one or more sensors includes a flowcell with a fixed pathlength.

In some embodiments of any of the bioprocessing systems described herein, at least one of the one or more sensors includes a flowcell with a variable pathlength.

In some embodiments of any of the bioprocessing systems described herein, a second unit operation is integrated upstream of the bioprocessing system, such that the second unit operation is in fluid communication with the inlet conduit of the bioprocessing system.

In some embodiments, the second unit operation is selected from the group consisting of a bioreactor unit operation, a cell removal unit operation, a cell retention unit operation, a chromatography unit operation, a viral inactivation unit operation, a virus removal unit operation, an ultrafiltration unit operation, and a diafiltration unit operation.

In some embodiments of any of the bioprocessing systems described herein, a second unit operation is integrated downstream of the bioprocessing system, such that the second unit operation is in fluid communication with the permeate conduit of the bioprocessing system.

In some embodiments of any of the bioprocessing systems described herein, a second unit operation is integrated downstream of the bioprocessing system, such that the second unit operation is in fluid communication with the retentate conduit of the bioprocessing system.

In some embodiments of any of the bioprocessing systems described herein, the second unit operation is selected from the group consisting of a chromatography unit operation, a viral inactivation unit operation, a virus removal unit operation, an ultrafiltration unit operation, a diafiltration unit operation, and a formulation unit operation.

As used herein, the word “a” before a noun represents one or more of the particular noun. For example, the phrase “a mammalian cell” represents “one or more mammalian cells.”

The terms “tangential flow filtration unit” or “TFF unit” are art-known and mean a device that includes at least one housing (such as a cylinder) and at least one cross-flow (tangential) filter positioned in the housing such that a large portion of the filter's surface is positioned parallel to the flow of a fluid (e.g., a cell culture) through the unit. TFF units are well-known in the art and are commercially available. The housing can include a first inlet/outlet and a second inlet/outlet positioned, e.g., to allow fluid to pass through the first inlet/outlet, cross the at least one cross-flow filter, and through the second inlet/outlet. In some examples, a bioprocessing system can include multiple TFF units, e.g., connected in series and/or in parallel. For example, a bioprocessing system that includes two or more TFF units can include fluid conduits fluidly connecting neighboring pairs of TFF units in the system. In other examples, a bioprocessing system can include two or more sets of two or more TFF units fluidly connected by fluid conduits. Any of the TFF units described herein or known in the art are capable of receiving fluid in a first flow direction and a second flow direction.

The term “cross-flow filter” or “tangential filter” is art known and means a filter that is designed such that it can be positioned in a TFF unit such that a large portion of the filter's surface is parallel to the flow of a fluid (e.g., a fluid including a recombinant therapeutic protein). For example, a cross-flow filter can have any shape that allows for tangential flow filtration, e.g., a tubular or rectangular shape. Particularly useful cross-flow filters are designed to result in a low amount of fluid turbulence or shear stress in the fluid when the fluid is flowed (e.g., unidirectionally flowed to bidirectionally flowed) across the surface of the cross-flow filter. Cross-flow filters are commercially available, e.g., from Sartorius, MembraPure, Millipore, and Pall Corporation.

The term “ultrafiltration” and “UF,” and like terms refer to using synthetic semi-permeable membranes, with appropriate physical and chemical properties, to segregate between molecules in the mixture, primarily on the basis of molecular size and shape, and accomplish either the separation of different molecules, or accomplish concentration of like molecules.

The term “diafiltration” and “DF,” and like terms refer to using an ultrafiltration membrane to remove, replace, or lower the concentration of salts or solvents from solutions or mixtures containing proteins, peptides, nucleic acids, or other biomolecules.

The term “permeate stream” or “permeate” means the stream of fluid that exits the TFF unit after passing across the TFF filter. Fluid components that are selectively retained by the filter will not be present in the permeate. In some embodiments (e.g., ultrafiltration or diafiltration), the permeate will not contain the recombinant therapeutic protein. In some embodiments (e.g., viral filtration or microfiltration), the permeate will contain the recombinant therapeutic protein but will not contain other impurities (e.g., viral particles or cell debris) present in the TFF feed material.

The term “retentate stream” or “retentate” means the stream of fluid that that exits the TFF unit without passing across the TFF filter. Fluid components that are selectively retained by the filter will be present in the retentate. In some embodiments (e.g., ultrafiltration), fluid components that are selectively retained by the filter will be enriched in the retentate.

The term “transmembrane pressure” or “TMP” refers to the average applied pressure from the feed to the filtrate side of the membrane calculated as TMP [bar]=[(P_{F}+P_{R})/2]−P_{P}. where P_{F} is the feed pressure, P_{R} is the retentate pressure, and P_{P} is the permeate pressure.

The term “dynamic pump” or “rotodynamic pump” means a type of velocity pump in which kinetic energy is continuously imparted to the pumped fluid by means of a rotating impeller, propeller, or rotor.

The term “centrifugal pump” means a dynamic pump that uses bladed impellers with essentially radial output to transfer rotational mechanical energy to the fluid primarily by increasing the fluid kinetic energy (angular momentum) and also increasing potential energy (static pressure).

The term “mammalian cell” means any cell from or derived from any mammal (e.g., a human, a hamster, a mouse, a green monkey, a rat, a pig, a cow, or a rabbit). For example, a mammalian cell can be an immortalized cell. In some embodiments, the mammalian cell is a differentiated cell. In some embodiments, the mammalian cell is an undifferentiated cell. Non-limiting examples of mammalian cells are described herein. Additional examples of mammalian cells are known in the art.

The term “unit operation” is a term of art and, when used as a verb, means a functional step that can be performed in a process of manufacturing a therapeutic protein drug substance from a liquid culture medium. For example, a unit of operation, when used as a verb, can be filtering (e.g., removal of contaminant bacteria, yeast viruses, or mycobacteria, and/or particular matter from a fluid containing a recombinant therapeutic protein), capturing, epitope tag removal, purifying, holding or storing, polishing, viral inactivating, adjusting the ionic concentration and/or pH of a fluid containing the recombinant therapeutic protein, and removing unwanted salts.

The term “unit operation” is a term of art and, when used as a noun, means instrumentation that can be used to perform a functional step in a process of manufacturing a therapeutic protein drug substance from a liquid culture medium. For example, a unit operation, when used as a noun, can include a chromatography system (e.g., a single-column or a multi-column chromatography system, a periodic countercurrent chromatography system, a filtration unit (e.g., a tangential flow filtration (TFF) unit), and/or a holding tank. Such unit operation may include, without limitation, a bioreactor unit operation, a cell removal unit operation, a cell retention unit operation, a tangential flow filtration unit operation, a chromatography unit operation, a viral inactivation unit operation, a virus removal filtration unit operation, an ultrafiltration unit operation, a diafiltration unit, or a formulation unit operation.

The term “continuous unit operation” means a unit operation which continuously feeds fluid through at least part of the system.

The term “continuous process” means a process which continuously feeds fluid through at least a part of the system.

The term “integrated process” means a process which is performed using structural elements that function cooperatively to achieve a specific result (e.g., the generation of a therapeutic protein drug substance from a liquid culture medium).

The term “bioprocessing system” refers to system for making, producing, or manufacturing a biologic from a liquid culture medium. Bioprocessing system may have one or more unit operations.

The term “upstream” refers to the performance of one or more unit operation(s) prior to the timing of the performance of one or more other unit operation(s). The term “downstream” refers to the performance of one or more unit operation(s) after the timing of the performance of one or more other unit operation(s).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-1F show overviews and control strategies of the first single-pass continuous tangential flow filtration (SP-TFF) or ultrafiltration 1 (UF1), single-pass continuous diafiltration (SP-DF), and the second single-pass continuous tangential flow filtration (SP-TFF) or ultrafiltration 2 (UF2) operations, respectively. As seen in FIG. 1A, the fluid containing the recombinant therapeutic protein is delivered from the surge vessel into the first ultrafiltration (UF) unit. The in-line flowmeters, pressure sensors, and refractive index sensors in the feed, retentate, and permeate streams measure in real-time the flowrate, pressure, and concentration values, respectively. The back pressure regulator (BPR) installed on the retentate stream controls the pressure exerted on the filter thereby concentrating the product. In FIG. 1B, the booster pump channels the emerging concentrated product stream into the SP-DF unit. Simultaneously, the diafiltration (DF) buffer enters the DF unit. The BPR maintains consistent retentate concentration by adjusting the permeate flux through the filter based on real-time fluid properties measured using the in-line sensors. FIG. 1C shows the final UF2 operation, which involves concentrating the recombinant therapeutic protein to a value higher than the final DS using a single UF unit. FIG. 1D shows the control strategy for continuous single-pass ultrafiltration (SP-TFF) operation, wherein a concentration controller is implemented to control the retentate stream concentration based on the real-time 1) feed and retentate concentrations, and 2) the feed and permeate fluxes. FIGS. 1E and 1F show the control strategies for continuous single-pass diafiltration (SP-DF) operation. FIG. 1E shows the strategy for implementation of a concentration controller that controls the retentate stream concentration based on the real-time 1) feed and retentate concentrations, and 2) the feed and permeate fluxes. FIG. 1F shows the control strategy for the implementation of a buffer addition controller that controls the fixed volumetric addition of buffer to feed based on 1) the calculated buffer flowrate, and 2) the real-time buffer flowrate.

FIGS. 2A-2D pertain to the first ultrafiltration (UF1) operation. FIG. 2A is a graph showing the recombinant therapeutic protein concentration (g/L) as a function of time (days) following the first ultrafiltration operation. FIG. 2B is a graph showing flux/TMP values as a function of time (days). FIG. 2C is a graph showing the permeate flux (LMH) as a function of time (days). FIG. 2D is a graph showing the permeate flux as a function of the applied transmembrane pressure (psi).

FIGS. 3A-311 are plots showing the performance of the continuous diafiltration operation. FIG. 3A is a graph showing the targeted diafiltration (DF) retentate concentration (g/L) over time (days). FIG. 3B is a graph showing the achieved and the targeted diavolumes over time (days). FIG. 3C is a graph showing the ratio of the diafiltration (DF) buffer addition rate to the permeate generated rate over time (days). FIG. 3D is a graph showing flux/TMP values over time (days). FIG. 3E shows the permeate volumetric flux (LMH) during diafiltration over time (days). FIG. 3F is a graph showing the effect of the permeate flux (LMH) on the transmembrane pressure (psi). FIG. 3G is a graph showing the effects of a booster pump used in combination with a back pressure regulator on retentate concentration (g/L) over time (days). FIG. 3H is a graph showing the effects of a booster pump used in combination with a back pressure regulator on the retentate pressure (psi) over the pressure of the back pressure regulator (psi).

FIGS. 4A-4D are graphs showing the last SP-TFF ultrafiltration operation (UF2) performance for the integrated manufacturing of the recombinant therapeutic protein. FIG. 4A is a graph showing the retentate protein concentration following ultrafiltration over time (days). FIG. 4B is a graph showing the filter performance (flux/TMP) during the operation over time (days). FIG. 4C is a graph showing the permeate flux (LMH) as function of time (days). FIG. 4D is a graph showing the permeate flux as a function of transmembrane pressure (psi).

### DETAILED DESCRIPTION

Provided herein are methods of processing a fluid and bioprocessing systems. Non-limiting aspects of these methods and bioprocessing systems are described herein.

### Methods of Processing a Fluid

Provided herein methods of processing a fluid comprising a recombinant therapeutic protein, comprising: (a) providing a bioprocessing system comprising: (i) a unit operation, (ii) at least one inlet on the unit operation, (iii) at least one outlet on the unit operation, (iv) an inlet conduit in fluid communication with at least one inlet of the unit operation, (v) an outlet conduit in fluid communication with at least one outlet of the unit operation, (vi) a back pressure regulator disposed in the outlet conduit, and (vii) a booster pump disposed in the outlet conduit that follows downstream of the back pressure regulator; (b) flowing the fluid comprising the recombinant therapeutic protein through the inlet conduit and into the at least one inlet of the unit operation, processing the fluid within the unit operation to obtain a processed fluid, and then flowing a portion of the processed fluid into the outlet conduit and (c) maintaining a target flow rate and/or pressure through the outlet conduit using the back pressure regulator and the booster pump.

Provided herein methods of processing a fluid comprising a recombinant therapeutic protein, the method comprising: (a) providing a bioprocessing system comprising: (i) a tangential flow filtration (TFF) unit with a permeate side and a retentate side, (ii) at least one inlet on the TFF unit, (iii) at least two outlets on the TFF unit, where one of the at least two outlets is on the permeate side of the TFF unit and one of the at least two outlets is on the retentate side of the TFF unit, (iv) an inlet conduit in fluid communication with at least one inlet of the TFF unit, (v) a permeate conduit in fluid communication with the outlet on the permeate side of the TFF unit, (vi) a retentate conduit in fluid communication with the outlet on the retentate side of the TFF unit, (vii) a back pressure regulator disposed in the permeate or retentate conduit, and (viii) a booster pump disposed in the permeate or retentate conduit that follows downstream of the back pressure regulator; (b) flowing the fluid comprising the recombinant therapeutic protein through the inlet conduit and into the at least one inlet of the TFF unit, and separating the fluid into retentate and permeate streams based on the pore size or molecular weight cutoff of the TFF unit, and then flowing those permeate and retentate streams into the permeate and retentate conduits, respectively; and (c) maintaining a target flow rate and/or pressure through the permeate or retentate conduit using the back pressure regulator and the booster pump.

Provided herein methods of processing a fluid comprising a recombinant therapeutic protein, the method comprising: (a) providing a bioprocessing system comprising: (i) a tangential flow filtration (TFF) unit with a permeate side and a retentate side, (ii) at least one inlet on the TFF unit, (iii) at least two outlets on the TFF unit, where one of the at least two outlets is on the permeate side of the TFF unit and one of the at least two outlets is on the retentate side of the TFF unit, (iv) an inlet conduit in fluid communication with at least one inlet of the TFF unit, (v) a permeate conduit in fluid communication with the outlet on the permeate side of the TFF unit, (vi) a retentate conduit in fluid communication with the outlet on the retentate side of the TFF unit, (vii) a back pressure regulator disposed in the permeate conduit, and (viii) a booster pump disposed in the permeate conduit that follows downstream of the back pressure regulator; (b) flowing the fluid comprising the recombinant therapeutic protein through the inlet conduit and into the at least one inlet of the TFF unit, and separating the fluid into retentate and permeate streams based on the pore size or molecular weight cutoff of the TFF unit, and then flowing those permeate and retentate streams into the permeate and retentate conduits, respectively; and (c) maintaining a target flow rate and/or pressure through the permeate conduit using the back pressure regulator and the booster pump.

Provided herein methods of processing a fluid comprising a recombinant therapeutic protein, the method comprising: (a) providing a bioprocessing system comprising: (i) a tangential flow filtration (TFF) unit with a permeate side and a retentate side, (ii) at least one inlet on the TFF unit, (iii) at least two outlets on the TFF unit, where one of the at least two outlets is on the permeate side of the TFF unit and one of the at least two outlets is on the retentate side of the TFF unit, (iv) an inlet conduit in fluid communication with at least one inlet of the TFF unit, (v) a permeate conduit in fluid communication with the outlet on the permeate side of the TFF unit, (vi) a retentate conduit in fluid communication with the outlet on the retentate side of the TFF unit, (vii) a back pressure regulator disposed in the retentate conduit, and (viii) a booster pump disposed in the retentate conduit that follows downstream of the back pressure regulator; (b) flowing the fluid comprising the recombinant therapeutic protein through the inlet conduit and into the at least one inlet of the TFF unit, and separating the fluid into retentate and permeate streams based on the pore size or molecular weight cutoff of the TFF unit, and then flowing those permeate and retentate streams into the permeate and retentate conduits, respectively; and (c) maintaining a target flow rate and/or pressure through the permeate or retentate conduit using the back pressure regulator and the booster pump.

Provided herein methods of processing a fluid comprising a recombinant therapeutic protein, the method comprising: (a) providing a bioprocessing system comprising: (i) a tangential flow filtration (TFF) unit with a permeate side and a retentate side, (ii) at least one inlet on the TFF unit, (iii) at least two outlets on the TFF unit, where one of the at least two outlets is on the permeate side of the TFF unit and one of the at least two outlets is on the retentate side of the TFF unit, (iv) an inlet conduit in fluid communication with at least one inlet of the TFF unit, (v) a permeate conduit in fluid communication with the outlet on the permeate side of the TFF unit, (vi) a retentate conduit in fluid communication with the outlet on the retentate side of the TFF unit, (vii) a back pressure regulator disposed in the retentate conduit, and (viii) a booster pump disposed in the retentate conduit that follows downstream of the back pressure regulator; (b) flowing the fluid comprising the recombinant therapeutic protein through the inlet conduit and into the at least one inlet of the TFF unit, and separating the fluid into retentate and permeate streams based on pore size or molecular weight cutoff of the TFF unit, and then flowing those permeate and retentate streams into the permeate and retentate conduits, respectively; and (c) maintaining a target flow rate and/or pressure through the permeate or retentate conduit using the back pressure regulator and the booster pump; wherein the TFF unit has an average pore size from about 15 nm to about 35 nm or about 1 μm to about 10 μm.

Provided herein methods of processing a fluid comprising a recombinant therapeutic protein, the method comprising: (a) providing a bioprocessing system comprising: (i) a tangential flow filtration (TFF) unit with a permeate side and a retentate side, (ii) at least one inlet on the TFF unit, (iii) at least two outlets on the TFF unit, where one of the at least two outlets is on the permeate side of the TFF unit and one of the at least two outlets is on the retentate side of the TFF unit, (iv) an inlet conduit in fluid communication with at least one inlet of the TFF unit, (v) a permeate conduit in fluid communication with the outlet on the permeate side of the TFF unit, (vi) a retentate conduit in fluid communication with the outlet on the retentate side of the TFF unit, (vii) a back pressure regulator disposed in the retentate conduit, and (viii) a booster pump disposed in the retentate conduit that follows downstream of the back pressure regulator; (b) flowing the fluid comprising the recombinant therapeutic protein through the inlet conduit and into the at least one inlet of the TFF unit, and separating the fluid into retentate and permeate streams based on pore size or molecular weight cutoff of the TFF unit, and then flowing those permeate and retentate streams into the permeate and retentate conduits, respectively; and (c) maintaining a target flow rate and/or pressure through the permeate or retentate conduit using the back pressure regulator and the booster pump; wherein the TFF unit has a molecular weight cutoff from about 1 kDa to about 100 kDa, and optionally, recombinant therapeutic protein concentration within the retentate conduit is maintained constant at about 5 to about 250 g/L. Provided herein methods of processing a fluid comprising a recombinant therapeutic protein, the method comprising: (a) providing a bioprocessing system comprising: (i) a tangential flow filtration (TFF) unit with a permeate side and a retentate side, (ii) at least one inlet on the TFF unit, (iii) at least two outlets on the TFF unit, where one of the at least two outlets is on the permeate side of the TFF unit and one of the at least two outlets is on the retentate side of the TFF unit, (iv) an inlet conduit in fluid communication with at least one inlet of the TFF unit, (v) a permeate conduit in fluid communication with the outlet on the permeate side of the TFF unit, (vi) a retentate conduit in fluid communication with the outlet on the retentate side of the TFF unit, (vii) a back pressure regulator disposed in the retentate conduit, and (viii) a booster pump disposed in the retentate conduit that follows downstream of the back pressure regulator, (ix) at least three sensors, wherein one of the at least three sensors disposed in the inlet conduit and one disposed in the permeate conduit; one sensor disposed in the retentate conduit; and (x) a feedback and feed-forward controller connected to the back pressure regulator; (b) flowing the fluid comprising the recombinant therapeutic protein through the inlet conduit and into the at least one inlet of the TFF unit, and separating the fluid into retentate and permeate streams based on pore size or molecular weight cutoff of the TFF unit, and then flowing those permeate and retentate streams into the permeate and retentate conduits, respectively; and (c) maintaining a target flow rate and/or pressure through the permeate or retentate conduit using the back pressure regulator and the booster pump; wherein the TFF unit has a molecular weight cutoff from about 1 kDa to about 100 kDa, and optionally, the recombinant therapeutic protein concentration within the retentate conduit is maintained constant in real-time at about 5 to 250 g/L by controlling pressure on the back pressure regulator.

In some embodiments, the bioprocessing system further comprises a cell removal unit operation, a cell retention unit operation, a tangential flow filtration unit operation, a chromatography unit operation, a viral inactivation unit operation, a virus removal unit operation, or a formulation unit operation.

In some embodiments, the method further comprises, prior to step (a), performing one or more unit operations on the recombinant therapeutic protein such that the one or more unit operations are integrated and in fluid communication with one or more of the inlets on the bioprocessing system described in step (a), and the one or more unit operations are selected from the group consisting of a bioreactor unit operation, a cell removal unit operation, a cell retention unit operation, a chromatography unit operation, a viral inactivation unit operation, a virus removal unit operation, an ultrafiltration unit operation, a diafiltration unit operation and a formulation unit.

In some embodiments, the method further comprises, after step (c), performing one or more second unit operation(s), wherein the one or more second unit operation(s) is/are integrated downstream of the bioprocessing system, such that one of the one or more second unit operation(s) is in fluid communication with the permeate conduit of the bioprocessing system.

In some embodiments, the method further comprises, after step (c), performing one or more second unit operation(s), such that one of the one or more second unit operation(s) is in fluid communication with the retentate conduit of the bioprocessing system.

In some embodiments, the method further comprises, after step (c), performing one or more second unit operation(s), wherein the one or more second unit operation(s) is/are integrated downstream of the bioprocessing system, wherein the one or more second unit operation(s) are selected from the group consisting of a chromatography unit operation, a viral inactivation unit operation, a virus removal unit operation, an ultrafiltration unit operation, a diafiltration unit operation, and a formulation unit operation.

In some embodiments, the TFF unit includes one, two, three, four, five, six, seven, eight, nine, ten, eleven, or twelve tangential filters. In some examples of these methods, the TFF unit can include one or more tangential filter(s) having a surface area of about 50 cm² to about 10 m², (e.g., about 50 cm² to about 0.1 m², about 50 cm² to about 0.5 m², about 50 cm² to about 1 m², about 50 cm² to about 5 m², about 50 cm² to about 10 m², about 0.1 m² to about 0.5 m², about 0.1 m² to about 1 m², about 0.1 m² to about 5 m², about 0.1 m² to about 10 m², about 0.5 m² to about 1 m², about 0.5 m² to about 5 m², about 0.5 m² to about 10 m², about 1 m² to about 5 m², about 1 m² to about 10 m², or about 5 m² to about 10 m²). In some embodiments, the TFF unit is comprised of one or more filters each with filter areas of about 0.1 m² to about 5 m².

In some embodiments, the TFF unit (one or more tangential filters in the TFF unit) has an average pore size from about 10 nm to about 100 nm, e.g., about 10 nm to about 15 nm, about 10 nm to about 20 nm, about 10 nm to about 35 nm, about 10 nm to about 50 nm, about 10 nm to about 100 nm, about 15 nm to about 20 nm, about 15 nm to about 35 nm, about 15 nm to about 50 nm, about 15 nm to about 100 nm, about 20 nm to about 35 nm, about 20 nm to about 50 nm, about 20 nm to about 100 nm, about 35 nm to about 50 nm, about 35 nm to about 100 nm, or about 50 nm to about 100 nm).

For example, a TFF unit comprising one or more tangential filters having an average pore size of about 10 nm to about 100 nm can be used to perform viral filtration.

In some embodiments, the TFF unit (one or more tangential filters in the TFF unit) has an average pore size from about 0.1 μm to about 10 μm (e.g., about 0.1 μm to about 0.2 μm, about 0.1 μm to about 0.45 μm, about 0.1 μm to about 0.65 μm, about 0.1 μm to about 1 μm, about 0.1 μm to about 5 μm, about 0.1 μm to about 10 μm, about 0.2 μm to about 0.45 μm, about 0.2 μm to about 0.65 μm, about 0.2 μm to about 1 μm, about 0.2 μm to about 5 μm, about 0.2 μm to about 10 μm, about 0.45 μm to about 0.65 μm, about 0.45 μm to about 1 μm, about 0.45 μm to about 5 μm, about 0.45 μm to about 10 μm, about 0.65 μm to about 1 μm, about 0.65 μm to about 5 μm, about 0.65 μm to about 10 μm, about 1 μm to about 5 μm, about 1 μm to about 10 μm, or about 5 μm to about 10 μm). In some embodiments, the TFF unit has an average pore size from about 10 nm to about 100 nm. In some embodiments, the TFF unit has an average pore size from about 15 nm to about 35 nm.

For example, a TFF unit comprising one or more tangential filters having an average pore size of about 0.1 μm to about 10 μm can be used to perform microfiltration.

In some embodiments, the one or more tangential filters in the TFF unit can have a molecular weight cut-off of about 1 kDa to about 1000 kDa (e.g., about 1 kDa to about 10 kDa, about 1 kDa to about 30 kDa, about 1 kDa to about 50 kDa, about 1 kDa to about 100 kDa, about 1 kDa to about 300 kDa, about 1 kDa to about 1000 kDa, about 10 kDa to about 30 kDa, about kDa to about 50 kDa, about 10 kDa to about 100 kDa, about 10 kDa to about 300 kDa, about kDa to about 1000 kDa, about 30 kDa to about 50 kDa, about 30 kDa to about 100 kDa, about kDa to about 300 kDa, about 30 kDa to about 1000 kDa, about 50 kDa to about 100 kDa, about 50 kDa to about 300 kDa, about 50 kDa to about 1000 kDa, about 100 kDa to about 300 kDa, about 100 kDa to about 1000 kDa, or about 300 kDa to about 1000 kDa). In some embodiments, the TFF unit has a molecular weight cutoff from about 10 kDa to about 100 kDa.

For example, a TFF unit comprising one or more tangential filters having a molecular weight cutoff of about 1 kDa to about 1000 kDa can be used to perform ultrafiltration and/or dialfiltration.

In some embodiments, the TFF unit includes one or more tangential filters made of, e.g., cellulose (e.g., regenerated cellulose or cuprammonium regenerated cellulose), polyethersulfone, polysulfone, polyvinylidene fluoride, or mixed cellulose ester. Non-limiting examples of one or more tangential filters that can be used in the TFF unit are commercially available from Millipore (e.g., Ultracel® and Biomax®). Additional commercial sources of one or more tangential filters that can be used in the TFF unit are known in the art.

In some embodiments, the recombinant therapeutic protein in the fluid (also referred as feed stream) has a concentration from about 0.1 g/L to about 300 g/L (e.g., about 0.1 g/L to about 5 g/L, about 0.1 g/L to about 25 g/L, about 0.1 g/L to about 50 g/L, about 0.1 g/L to about 100 g/L, about 0.1 g/L to about 150 g/L, about 0.1 g/L to about 225 g/L, about 0.1 g/L to about 300 g/L, about 5 g/L to about 25 g/L, about 5 g/L to about 50 g/L, about 5 g/L to about 100 g/L, about 5 g/L to about 150 g/L, about 5 g/L to about 225 g/L, about 5 g/L to about 300 g/L, about 25 g/L to about 50 g/L, about 25 g/L to about 100 g/L, about 25 g/L to about 150 g/L, about 25 g/L to about 225 g/L, about 25 g/L to about 300 g/L, about 50 g/L to about 100 g/L, about 50 g/L to about 150 g/L, about 50 g/L to about 225 g/L, about 50 g/L to about 300 g/L, about 100 g/L to about 150 g/L, about 100 g/L to about 225 g/L, about 100 g/L to about 300 g/L, about 150 g/L to about 225 g/L, about 150 g/L to about 300 g/L, or about 225 g/L to about 300 g/L).

In some embodiments, the recombinant therapeutic protein in the permeate stream has a concentration from about 0.1 g/L to about 100 g/L (e.g., about 0.1 g/L to about 1 g/L, about 0.1 g/L to about 5 g/L, about 0.1 g/L to about 10 g/L, about 0.1 g/L to about 25 g/L, about 0.1 g/L to about 100 g/L, about 1 g/L to about 5 g/L, about 1 g/L to about 10 g/L, about 1 g/L to about 25 g/L, about 1 g/L to about 100 g/L, about 5 g/L to about 10 g/L, about 5 g/L to about 25 g/L, about 5 g/L to about 100 g/L, about 10 g/L to about 25 g/L, about 10 g/L to about 100 g/L, or about 25 g/L to about 100 g/L). In some embodiments, the recombinant therapeutic protein in the permeate stream has a concentration from about 0.1 g/L to about 25 g/L. In some embodiments, there is no recombinant therapeutic protein in the permeate stream.

In some embodiments, the recombinant therapeutic protein in the retentate stream has a concentration from about 0.1 g/L to about 300 g/L (e.g., about 0.1 g/L to about 5 g/L, about 0.1 g/L to about 25 g/L, about 0.1 g/L to about 50 g/L, about 0.1 g/L to about 100 g/L, about 0.1 g/L to about 150 g/L, about 0.1 g/L to about 225 g/L, about 0.1 g/L to about 300 g/L, about 5 g/L to about 25 g/L, about 5 g/L to about 50 g/L, about 5 g/L to about 100 g/L, about 5 g/L to about 150 g/L, about 5 g/L to about 225 g/L, about 5 g/L to about 300 g/L, about 25 g/L to about 50 g/L, about 25 g/L to about 100 g/L, about 25 g/L to about 150 g/L, about 25 g/L to about 225 g/L, about 25 g/L to about 300 g/L, about 50 g/L to about 100 g/L, about 50 g/L to about 150 g/L, about 50 g/L to about 225 g/L, about 50 g/L to about 300 g/L, about 100 g/L to about 150 g/L, about 100 g/L to about 225 g/L, about 100 g/L to about 300 g/L, about 150 g/L to about 225 g/L, about 150 g/L to about 300 g/L, or about 225 g/L to about 300 g/L). In some embodiments, the recombinant therapeutic protein in the retentate stream has a concentration from about 5 g/L to about 225 g/L.

In some examples of any of the methods described herein, step (b) includes flowing (e.g., continuously flowing) the fluid including the recombinant therapeutic protein through the inlet conduit and into the at least one inlet of the TFF unit at a rate of between about 0.1 mL/minute to about 10 L/minute (e.g., about 0.1 mL/minute to about 1 mL/minute, about 0.1 mL/minute to about 5 mL/minute, about 0.1 mL/minute to about 25 mL/minute, about 0.1 mL/minute to about 100 mL/minute, about 0.1 mL/minute to about 500 mL/minute, about 0.1 mL/minute to about 1 L/minute, about 0.1 mL/minute to about 10 L/minute, about 1 mL/minute to about 5 mL/minute, about 1 mL/minute to about 25 mL/minute, about 1 mL/minute to about 100 mL/minute, about 1 mL/minute to about 500 mL/minute, about 1 mL/minute to about 1 L/minute, about 1 mL/minute to about 10 L/minute, about 5 mL/minute to about 25 mL/minute, about 5 mL/minute to about 100 mL/minute, about 5 mL/minute to about 500 mL/minute, about 5 mL/minute to about 1 L/minute, about 5 mL/minute to about 10 L/minute, about 25 mL/minute to about 100 mL/minute, about 25 mL/minute to about 500 mL/minute, about 25 mL/minute to about 1 L/minute, about 25 mL/minute to about 10 L/minute, about 100 mL/minute to about 500 mL/minute, about 100 mL/minute to about 1 L/minute, about 100 mL/minute to about 10 L/minute, about 500 mL/minute to about 1 L/minute, about 500 mL/minute to about 10 L/minute, or about 1 L/minute to about 10 L/minute). In some embodiments, step (b) comprises continuously flowing the fluid at an average rate from about 1 mL/minute to about 1 L/minute.

In some embodiments, the fluid comprises a diafiltration medium. Non-limiting examples of a diafiltration medium can be, e.g., a physiologically acceptable buffer (e.g., phosphate buffered saline) or a buffer used to formulate the recombinant therapeutic protein, e.g., a buffer comprising one or more of glycine, phosphate, acetate, citrate, histidine, arginine, a bulking agent (e.g., a non-reducing carbohydrate, e.g., sucrose), or a surfactant (e.g., polysorbate 80, Triton X-100, and Tween).

In some embodiments, the methods described herein include the use of a holding tank fluidly connected to the inlet conduit, the permeate conduit, and/or the retentate conduit. In some embodiments, the methods described herein do not include the use of a holding tank fluidly connected to the inlet conduit, the permeate conduit, and/or the retentate conduit. In some embodiments, the methods described herein do not include the use of a holding tank at all.

In some embodiments, where the TFF unit comprises a viral filter, the method can further comprise recovering the permeate stream containing recombinant therapeutic protein from the permeate conduit.

In some embodiments, where the TFF unit comprises a microfilter, the method can further comprise recovering the permeate stream containing recombinant therapeutic protein from the permeate conduit.

In some embodiments, where the TFF unit is an ultrafiltration or diafiltration filter, the method can further comprise recovering the retentate stream containing recombinant therapeutic protein from the retentate conduit.

In some embodiments, the booster pump can be a dynamic pump (e.g., a centrifugal pump). In some embodiments, the dynamic pump is a centrifugal pump.

In some embodiments, the bioprocessing system further includes one or more sensor(s). In some embodiments, at least one of the one or more sensor(s) is disposed in the inlet conduit. In some embodiments, at least one of the one or more sensor(s) is disposed in the permeate conduit. In some embodiments, at least one of the one or more sensor(s) is disposed in the retentate conduit. In some embodiments, at least one of the one or more sensor(s) is disposed in the outlet conduit.

In some embodiments, the recombinant therapeutic protein concentration is measured using the one or more sensor(s) that has a sample time of less than about 2 minutes (e.g., less than about 90 seconds, less than about 60 seconds, less than about 45 seconds, less than about 30 seconds, less than about 25 seconds, less than about 20 seconds, less than about 15 seconds, less than about 10 seconds, or less than about 5 seconds)).

In some embodiments, the therapeutic protein concentration is measured using UV absorbance, refractive index, Fourier Transform Infrared spectroscopy (FITR), or Raman spectroscopy.

In some embodiments, the recombinant therapeutic protein concentration is measured using UV absorbance between about 200 nm to about 400 nm. In some embodiments, the recombinant therapeutic protein concentration is measured using UV absorbance, and wherein the UV absorbance is measured between about 260 nm to about 320 nm. In some embodiments, the UV absorbance is measured with a flowcell with a fixed pathlength. In some embodiments, the UV absorbance is measured with a flowcell with a variable pathlength.

In some embodiments, the bioprocessing system accomplishes a buffer exchange of from about 5 to about 15 fold volumes. In some embodiments, the bioprocessing system accomplishes a buffer exchange of from about 5-7 fold volumes.

In some embodiments, the flux to transmembrane pressure (TMP) is maintained at 0.5 to 1.0 for at least five days (e.g., at least six days, at least seven days, at least eight days, at least nine days, or at least ten days). In some embodiments, the flux to transmembrane pressure (TMP) is maintained at 0.6 to 0.7 for at least five days (e.g., at least six days, at least seven days, at least eight days, at least nine days, or at least ten days). In some embodiments, the flux to transmembrane pressure (TMP) is maintained at a constant value for at least five days (e.g., at least six days, at least seven days, at least eight days, at least nine days, or at least ten days).

In some embodiments, the method maintains the target concentration of the recombinant therapeutic protein in retentate stream of the TFF unit in real-time over a period of time. In some embodiments, the method maintains the target concentration of the recombinant therapeutic protein in retentate stream of the TFF unit in real-time over about 3 days to about 8 weeks. In some embodiments, the method maintains the target concentration of the recombinant therapeutic protein in retentate stream of the TFF unit in real-time over about 4 weeks.

### Exemplary Ultrafiltration Method

In a continuous format, the ultrafiltration operation is divided into two steps: ultrafiltration 1 (UF1) and ultrafiltration 2 (UF2) with an intermediate diafiltration (DF) step. The protein solution is fed as input to the UF1. The protein solution can be concentrated to an intermediate value suitable for diafiltration using two 0.5 m² ultrafiltration units in a series configuration. Such a configuration allows for maximum sweeping of the inlet stream thereby preventing concentration polarization and fouling on the membrane surface. Briefly, the entire UF1 operation is performed as follows: the inlet protein solution from the surge vessel (5L, Cercell) is pumped using a quaternary diaphragm pump (Quattroflow Fluid Systems, QF 150S, PSG Germany GmbH) to the inlet of a 0.5 m² single-pass tangential flow filtration (SP-TFF) unit (30 kDa molecular weight cut-off (MWCO), Pellicon capsule with Ultracel membrane, C-screen). The volumetric flowrate of the feed stream is adjusted automatically using a level-controller on the surge vessel. Subsequently, the retentate stream from the first UF1 unit is fed to the inlet of the second UF1 unit. In this entire process, a proportional-integral-derivative (PID) controller, which uses a combination of feedback and feed-forward control, is adopted to maintain the protein concentration in the retentate at the targeted value. The PID controller modulates the transmembrane pressure (TMP) on the retentate stream of the second UF1 unit using a back pressure regulator (BPR, Equilibar, Precision Fluid Control) based on the feed and permeate flux, and the feed and retentate concentration. These fluxes and concentrations are measured independently in real time by inline single-use flowmeters (Levitronix Technologies Inc.) and refractive index (RI) sensors (mPath IoR flowcell, Corporation Life Sciences), respectively. Finally, the retentate stream of the second UF1 unit is fed continuously to the downstream diafiltration unit via a booster pump (PuraLev i100SU, Levitronix Technologies Inc.), which is essentially a centrifugal pump. The booster pump minimizes the upstream pressure effects on the downstream operations and ensures a constant and uninterrupted flow to the downstream units. Thus, a combination of BPR and booster pump is essential for the robust control of UF1.

In the second continuous ultrafiltration step (UF2), the buffer-exchanged protein from the DF step is further concentrated to a value higher than that of the desired drug substance (DS). The operating principle and the control strategy are the same as in UF1. However, the primary difference between them is the use of one 0.5 m² single-pass tangential flow filtration (SP-TFF) unit (30 kDa molecular weight cut-off (MWCO), Pellicon capsule with Ultracel membrane, C-screen) in UF2 instead of two units as described in UF1. Following the concentration process, the booster pump directs the product to the formulation operation.

### Exemplary Diafiltration Method

The diafiltration operation involves the exchange of the starting buffer (300 mM sodium acetate, pH 4.5) of the concentrated protein solution from UF1 with 5-7 diavolumes (DVs) of DF buffer (10 mM sodium acetate, pH 4.5). To achieve in-line buffer exchange, the concentrated protein stream from the second UF1 unit is fed continuously (via the previously mentioned booster pump) to the inlet of a 1.6 m² single-pass diafiltration cassette (Hydrosart ultrafilter, 30 kDa molecular weight cut-off (MWCO), Sartorius). Simultaneously, a peristaltic pump (Watson-Marlow Inc.) feeds the DF buffer at a volumetric flowrate equivalent to 5 DVs (5 times the feed flowrate) based on the inline flowrate measurement (Levitronix Technologies Inc.) of the feed stream. The continuous DF operation is designed in such a way that the rate at which the DF buffer is added into the system is equal to the rate at which the permeate is removed from the system. This condition ensures that the protein concentration does not change during the process. The single-pass continuous DF strategy is achieved by the implementation of two controllers, namely 1) a concentration controller, maintaining consistent retentate concentration, and 2) a buffer addition controller, ensuring fixed volumetric addition of buffer to feed, i.e., the desired diavolumes. To maintain consistent retentate concentration, a proportional-integral-derivative (PID) controller, which uses a combination of feedback and feed-forward control, is implemented to control the TMP on the DF membrane. Similar to UF1, the controller modulates the TMP using a back pressure regulator based on inline measurements of the feed and permeate flux (single-use flowmeters, Levitronix Technologies Inc.) and the feed and retentate concentration (RI sensors, mPath IoR flowcell, Corporation Life Sciences). For achieving the desired diavolumes, the volumetric flowrate of the DF buffer is calculated in real time based on the feed flowrate and permeate flux through the DF cassette. Subsequently, another PID controller dynamically adjusts the set point to the peristaltic pump to vary the buffer addition rate. Both the controllers work in synergy to ensure robust control and operation of diafiltration in a continuous single-pass format. The final outlet stream is constantly fed to the next ultrafiltration unit (UF2) using a booster pump (PuraLev i100SU, Levitronix Technologies Inc.), for the same reasons outlined in UF1. As in UF1, the BPR and booster pump work together to enable robust, integrated operation of single-pass DF.

### Bioprocessing Systems

Also provided herein, a bioprocessing system comprising: (i) a unit operation; (ii) at least one inlet on the unit operation; (iii) at least one outlet on the unit operation; (iv) an inlet conduit in fluid communication with at least one inlet of the unit operation; (v) an outlet conduit in fluid communication with at least one outlet of the unit operation; (vi) a back pressure regulator disposed in the outlet conduit, and (vii) a booster pump disposed in the outlet conduit that follows downstream of the back pressure regulator, wherein: the bioprocessing system is configured to flow fluid comprising the recombinant therapeutic protein through the inlet conduit and into the at least one inlet of the unit operation, processing the fluid within the unit operation to obtain a processed fluid, and then flowing a portion of the processed fluid into the outlet conduit; and the back pressure regulator and the booster pump are configured to maintain a target flow rate and/or pressure through the outlet conduit.

Also provided herein, a bioprocessing system comprising: (i) a tangential flow filtration (TFF) unit having at least one inlet and at least two outlets, wherein one of the at least two outlets is on the permeate side of the TFF unit and one of the at least two outlets is on the retentate side of the TFF unit; (ii) an inlet conduit in fluid communication with the at least one inlet of the TFF unit; (iii) a permeate outlet conduit in fluid communication with the outlet on the permeate side of the TFF unit; (iv) a retentate conduit in fluid communication with the outlet on the retentate side of the TFF unit; (v) a back pressure regulator disposed in the permeate or retentate conduit, and (vi) a booster pump disposed in the permeate or retentate conduit that follows downstream of the back pressure regulator, wherein: the bioprocessing system is configured to flow fluid comprising a recombinant therapeutic protein through the inlet conduit and into the at least one inlet of the TFF unit, and separate the fluid into retentate and permeate streams based on the pore size or molecular weight cutoff of the TFF unit, and then flow those permeate and retentate streams into the permeate and retentate conduits; and the back pressure regulator and the booster pump are configured to maintain a target flow rate and/or pressure through the retentate conduit.

In some embodiments, the bioprocessing system comprises a cell removal unit operation, a cell retention unit operation, a chromatography unit operation, a viral inactivation unit operation, a virus removal unit operation, or a formulation unit operation.

In some embodiments, the bioprocessing system further comprises one or more additional unit operations such that the one or more additional unit operations are integrated and in fluid communication with one or more of the inlets on the unit operation, and the one or more additional unit operations are selected from the group consisting of a cell removal unit operation, a cell retention unit operation, a chromatography unit operation, a viral inactivation unit operation, a virus removal unit operation, an ultrafiltration unit operation, a diafiltration unit operation, or a formulation unit operation.

In some embodiments, the bioprocessing system comprises a second unit operation integrated upstream of the bioprocessing system, such that the second unit operation is in fluid communication with the inlet conduit of the bioprocessing system.

In some embodiments, the bioprocessing system comprises a second unit operation is selected from the group consisting of a bioreactor, a cell removal unit operation, a cell retention unit operation, a chromatography operation, a viral inactivation operation, a virus removal filtration operation, an ultrafiltration operation, and a diafiltration operation.

In some embodiments, the bioprocessing system comprises a second unit operation that is integrated downstream of the bioprocessing system, such that the second unit operation is in fluid communication with the permeate conduit of the bioprocessing system.

In some embodiments, the bioprocessing system comprises a second unit operation is integrated downstream of the bioprocessing system, such that the second unit operation is in fluid communication with the retentate conduit of the bioprocessing system.

In some embodiments, the bioprocessing system comprises a second unit operation is integrated downstream of the bioprocessing system, wherein the second unit operation is selected from the group consisting of a chromatography operation, a viral inactivation operation, a virus removal filtration operation, an ultrafiltration operation, a diafiltration operation, and a formulation operation.

In some embodiments of any of the bioprocessing systems described herein, the TFF unit includes one, two, three, four, five, six, seven, eight, nine, ten, eleven, or twelve tangential filters. In some examples of these bioprocessing systems, the TFF unit can include one or more tangential filter(s) having a surface area of about 50 cm² to about 10 m² (or any of the exemplary subranges of this range described herein).

### Back Pressure Regulator

In some embodiments, the methods and bioprocessing systems described herein include a back pressure regulator. A back pressure regulator can be used to maintain a stable and consistent pressure and/or flowrate and can be used under a variety of downstream pressure conditions, given that the pressure downstream of the back pressure regulator does not exceed the pressure upstream. Various types of back pressure regulators are known in the art and can be used in any of the methods and/or bioprocessing systems described herein (e.g., BP-2080 automatic back pressure regulator (Jasco, DE), ultra-low volume back pressure regulator (IDEX Health & Science LLC), M10BP Precision back pressure regulator (Fairchild)). See, e.g., Berger and Berger, J. Chromatogr. A 1218(16): 2320-2326, 2011, and Svensson et al., Sci. Rep. 12:569, 2022.

In some embodiments, the back pressure regulator is used in a bioprocessing system with a positive displacement pump and bypass valves.

### Booster Pump

In some embodiments of any of the methods and bioprocessing systems described herein, the booster pump is a dynamic pump (also known as a non-positive displacement pump). In some embodiments, the dynamic pump is a centrifugal pump or a propeller pump. A centrifugal pump transfers rotational energy from impellers into a fluid, increasing the velocity and pressure of the fluid. Unlike a positive displacement pump, a dynamic pump can operate at multiple pressures for a given flowrate and pump speed. Various types of dynamic pumps are known in the art and can be used in any of the methods and/or bioprocessing systems described herein (e.g., Puralev 600 MU (Levitronix, Switzerland), LKH UltraPure (Alfa Laval, Sweden)).

### Sensors

In some embodiments of any of the methods and bioprocessing systems described herein, one or more sensors can be used in the control of TFF unit. The relevant sensors used in the control algorithm measure the flowrate, pressure, and/or recombinant therapeutic protein concentration. The flowrate sensor(s) are inline flowmeters that measure the flowrate in real-time. The flow sensors can be installed, e.g., in the inlet of the TFF unit, the at least two outlets of the TFF unit, the inlet conduit, the permeate conduit, and/or in the retentate conduit.

The in-line pressure sensors placed downstream of the back pressure regulator and the booster pump change the rpm on the booster pump as the pressure varies, thereby ensuring continuous flow to the subsequent operations.

### Controller and Control Strategy

In some embodiments of any of the methods and bioprocessing systems described herein, one or more controllers is used to manipulate the process variable in such a way that the effect of disturbances can be minimized, and the process variable is as close as possible to its set point. A variety of control schemes including feedback control, feedforward control, cascade control, and model predictive control-based techniques can be used for robust control of a process.

In some embodiments, the bioprocessing system comprises a buffer addition controller which controls fixed volumetric addition of buffer to fluid (feed stream).

In some embodiments, the method accomplishes a buffer exchange at constant volume, constant concentration, or both.

In some embodiments, the bioprocessing system comprises a proportional-integral-derivative (PID) controller to control the transmembrane pressure of TFF unit and a buffer addition controller to control fixed volumetric addition of buffer to feed.

In one exemplary case, the control strategy uses a combination of feedback and feedforward control on two PID controller blocks to control the recombinant protein concentration in the retentate stream. The control algorithm modulates the transmembrane pressure (TMP) of the TFF unit by dynamically varying the pressure on the back pressure regulator based on the measured flowrates in any two of the feed (fluid in the inlet conduit), retentate stream (fluid in the retentate conduit), and permeate stream (fluid in the permeate conduit) and the measured concentration of the recombinant therapeutic protein in the feed, retentate, and permeate stream.

### Recombinant Therapeutic Proteins

The term “recombinant therapeutic protein” means a polypeptide that is not naturally encoded by an endogenous nucleic acid present within an organism (e.g., a mammal) and provides for a therapeutic effect when administered to a subject in need thereof. Examples of recombinant therapeutic proteins include enzymes (e.g., with one or more amino acid substitutions, deletions, insertions, or additions that result in an increase in stability and/or catalytic activity of the engineered enzyme), fusion proteins, antibodies (e.g., divalent antibodies, trivalent antibodies, or a diabody), and antigen-binding proteins that contain at least one recombinant scaffolding sequence.

Non-limiting examples of recombinant therapeutic proteins that can be processed using the methods provided herein include immunoglobulins (including light and heavy chain immunoglobulins, antibodies, or antibody fragments (e.g., any of the antibody fragments described herein), enzymes (e.g., a galactosidase (e.g., an alpha-galactosidase), Myozyme, or Cerezyme), proteins (e.g., human erythropoietin, tumor necrosis factor (TNF), or an interferon alpha or beta), or immunogenic or antigenic proteins or protein fragments (e.g., proteins for use in a vaccine). The recombinant therapeutic protein can be an engineered antigen-binding polypeptide that contains at least one multifunctional recombinant protein scaffold (see, e.g., the recombinant antigen-binding proteins described in Gebauer et al., Current Opin. Chem. Biol. 13:245-255, 2009; and U.S. Patent Application Publication No. 2012/0164066 (herein incorporated by reference in its entirety)). Non-limiting examples of recombinant therapeutic proteins that are antibodies include: panitumumab, omalizumab, abagovomab, abciximab, actoxumab, adalimumab, adecatumumab, afelimomab, afutuzumab, alacizumab, alacizumab, alemtuzumab, alirocumab, altumomab, amatuximab, amatuximab, anatumomab, anrukinzumab, apolizumab, arcitumomab, atinumab, tocilizumab, basilizimab, bectumomab, belimumab, bevacizumab, besilesomab, bezlotoxumab, biciromab, canakinumab, certolizumab, cetuximab, cixutumumab, daclizumab, denosumab, densumab, eculizumab, edrecolomab, efalizumab, efungumab, epratuzumab, ertumaxomab, etaracizumab, figitumumab, golimumab, ibritumomab tiuxetan, igovomab, imgatuzumab, infliximab, inolimomab, inotuzumab, labetuzumab, lebrikizumab, moxetumomab, natalizumab, obinutuzumab, oregovomab, palivizumab, panitumumab, pertuzumab, ranibizumab, rituximab, tocilizumab, tositumomab, tralokinumab, tucotuzumab, trastuzumab, veltuzumab, zalutumumab, and zatuximab. Additional examples of recombinant therapeutic antibodies that can be produced by the methods described herein are known in the art. Additional non-limiting examples of recombinant therapeutic proteins that can be produced by the present methods include: alglucosidase alfa, laronidase, abatacept, galsulfase, lutropin alfa, antihemophilic factor, agalsidase beta, interferon beta-la, darbepoetin alfa, tenecteplase, etanercept, coagulation factor IX, follicle stimulating hormone, interferon beta-la, imiglucerase, dornase alfa, epoetin alfa, insulin or insulin analogs, mecasermin, factov VIII, factor VIIa, anti-thrombin III, protein C, human albumin, erythropoietin, granulocute colony stimulating factor, granulocyte macrophage colony stimulating factor, interleukin-11, laronidase, idursuphase, galsulphase, α-1-proteinase inhibitor, lactase, adenosine deaminase, tissue plasminogen activator, thyrotropin alpha (e.g., Thyrogen®) and alteplase. Additional examples of recombinant therapeutic proteins that can be produced by the present methods include acid α-glucosidase, alglucosidase alpha (e.g., Myozyme® and Lumizyme®), α-L-iduronidase (e.g., Aldurazyme®), iduronate sulfatase, heparan N-sulfatase, galactose-6-sulfatase, acid β-galactosidase, β-glucoronidase, N-acetylglucosamine-1-phosphotransferase, α-N-acetylgalactosaminidase, acid lipase, lysosomal acid ceramidase, acid sphingomyelinase, β-glucosidase (e.g., Cerezyme® and Ceredase®), galactosylceramidase, α-galactosidase-A (e.g., Fabrazyme®), acid β-galactosidase, β-galactosidase, neuraminidase, hexosaminidase A, and hexosaminidase B.

### EXAMPLES

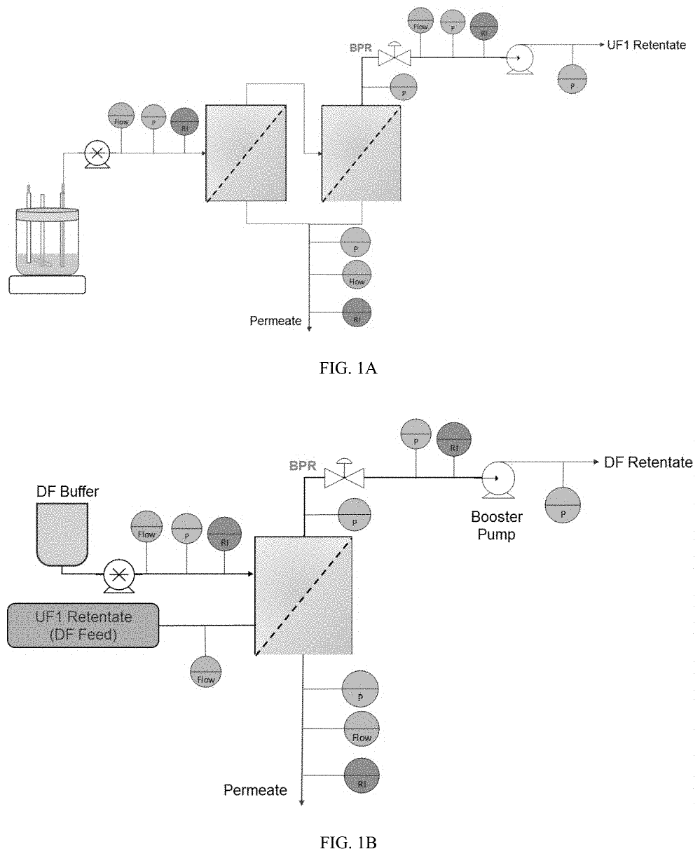

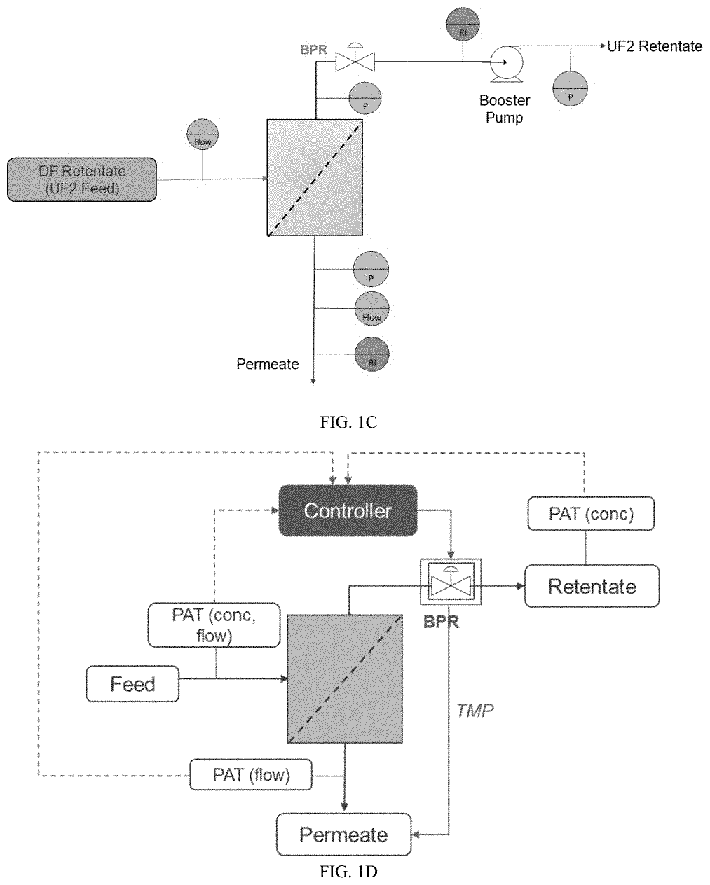

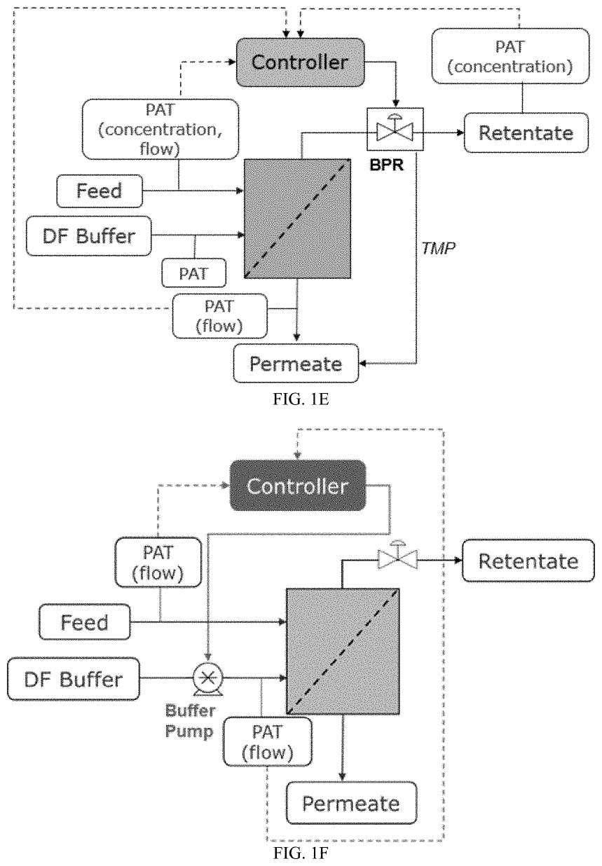

In one aspect, the UF and DF operations are divided into 3 steps: ultrafiltration 1 (UF1), diafiltration (DF), and the final ultrafiltration process (UF2). In ultrafiltration, a recombinant protein solution is concentrated by applying pressure to a semi-permeable membrane that retains the recombinant protein while allowing solvent and small molecular weight solutes to be removed from the solution. Analogous to ultrafiltration, a semi-permeable membrane is used in diafiltration to (i) remove or lower the salt concentration or (ii) exchange the buffer species of the protein solution. The original buffer species is exchanged with the new buffer species by adding them to the protein solution at a volumetric rate equivalent to the removal of the original species. This condition ensures that the protein solution volume and its concentration remain unchanged during the process. For integrated biomanufacturing of recombinant therapeutic proteins, UF and DF can be operated in a single-pass tangential flow filtration (SP-TFF) format (Casey et al., Journal of Membrane Science, 384(2): 82-88, 2011; U.S. Pat. No. 7,384,549; and Arunkumar et al., Journal of Membrane Science, 524: 20-32, 2017). The first ultrafiltration step (UF1) involves concentrating the protein to an intermediate value, which is favorable for diafiltration. The subsequent diafiltration step ensures buffer exchange of the protein solution. The final ultrafiltration step (UF2) concentrates the protein to the desired value. See FIGS. 1A-1C. The fluxes and concentrations are measured independently in real time by inline sensors and a controller modulates the transmembrane pressure (TMP) on the retentate stream using a back pressure regulator based on the feed and permeate flux, and the feed and retentate concentration to maintain consistent flow rate and retentate concentration (FIGS. 1D-1F).

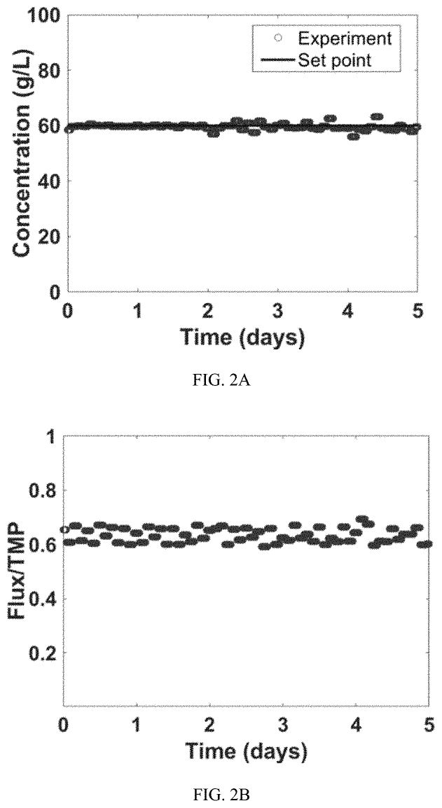

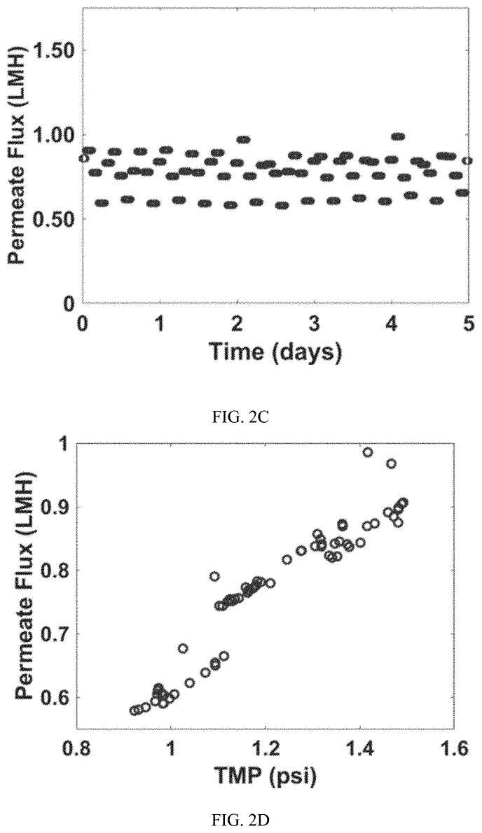

As an example of the UF1 operation, FIG. 2A shows the recombinant therapeutic protein concentration as a function of time following the first ultrafiltration operation. The recombinant therapeutic protein concentration remained relatively constant over time at around 60 g/L (FIG. 2A). In FIG. 2B, consistent flux/TMP values indicate filter integrity and absence of fouling throughout the course of operation. Throughout the course of operation, the permeate flux remained consistent (FIG. 2C), and the permeate flux correlated linearly with the applied transmembrane pressure (FIG. 2D). Taken together, the data in FIGS. 2A-2D demonstrated that the bioprocessing system regulated and maintained the recombinant therapeutic protein concentration and permeate flux while maintaining the integrity of the UF filter.

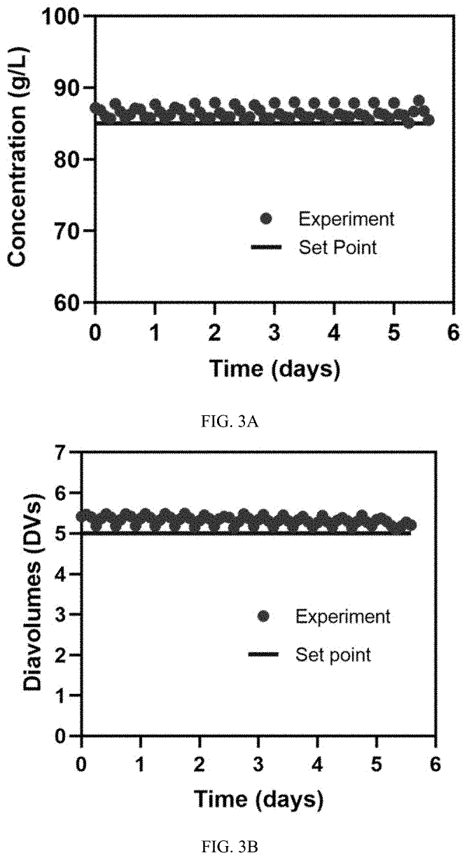

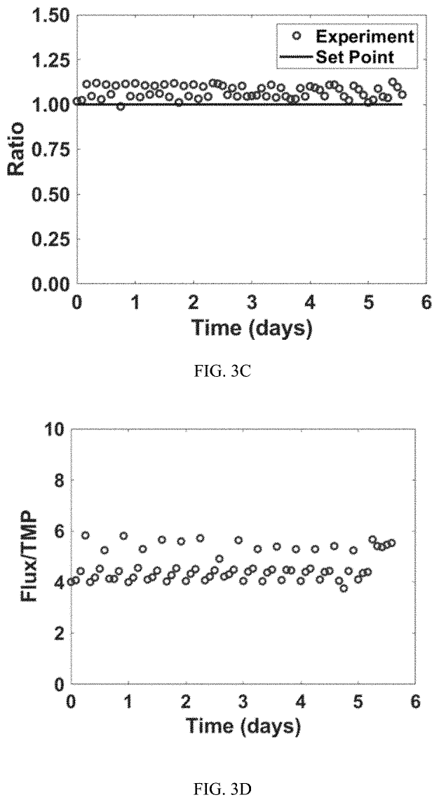

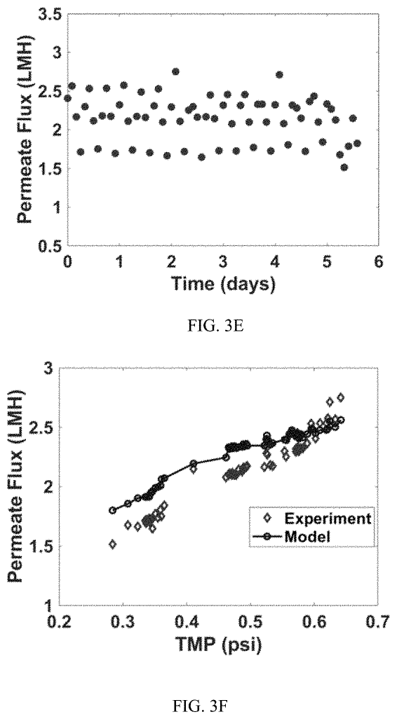

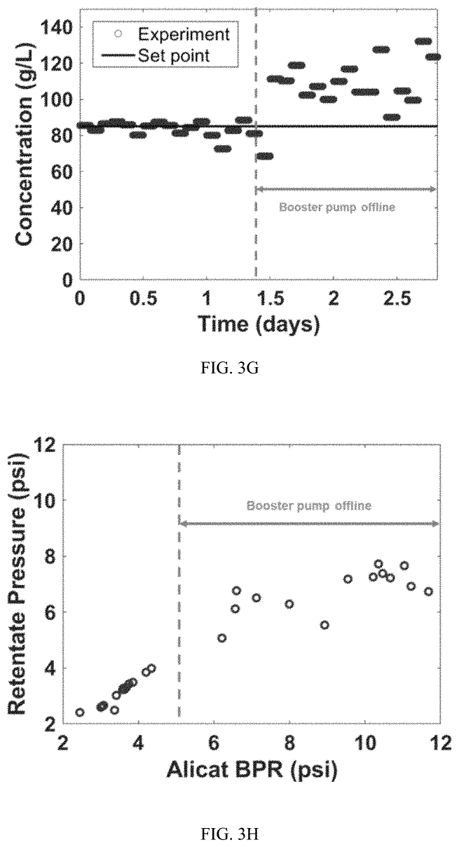

Performance of continuous diafiltration operation is shown in FIGS. 3A-3H. FIG. 3A demonstrates the ability to maintain the targeted DF retentate concentration over time, while FIG. 3B shows the achieved and the targeted diavolumes during the operation. Throughout the course of operation, the recombinant therapeutic protein concentration and diavolumes were maintained relatively constant over the course of six days. The ratio of the DF buffer addition rate to the permeate generated rate remained relatively constant over the course of six days (FIG. 3C). The DF filter performance, as determined by the flux/TMP, remained relatively constant over time (FIG. 3D). Throughout the course of operations, the permeate volumetric flux remained between 1.7 and 2.5 LMH (FIG. 3E). The effect of the permeate flux through the filter on the transmembrane pressure was modeled and experimentally determined (FIG. 3F). As shown in FIG. 3F, the experimentally determined permeate flux through the filter correlated with the transmembrane pressure. The data in FIGS. 3G and 3H demonstrate that a booster pump used in combination with the back pressure regulator decouples pressure effects and allows integration of subsequent operations. FIG. 3G shows the inability of the controller to maintain consistent DF retentate concentration when the booster pump is taken offline (˜days 1.4 to day 3). FIG. 3H shows that, with the booster pump offline, the back pressure regulator is no longer able to maintain consistent retentate pressure. In this case, the retentate pressure is determined by the resistance to flow downstream of the operation.

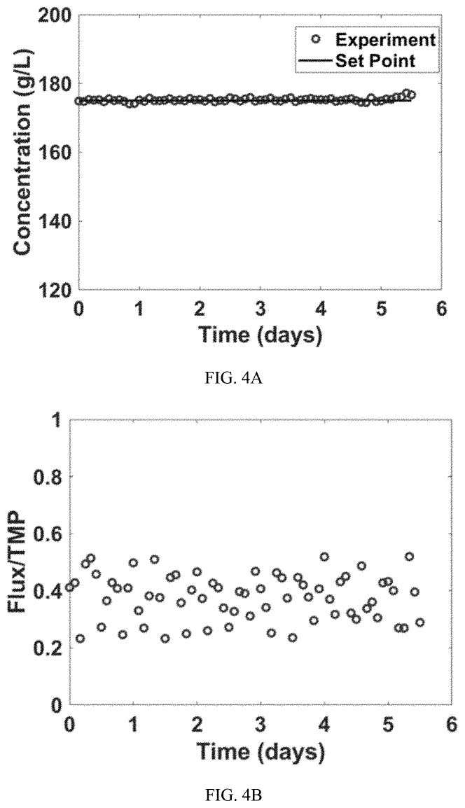

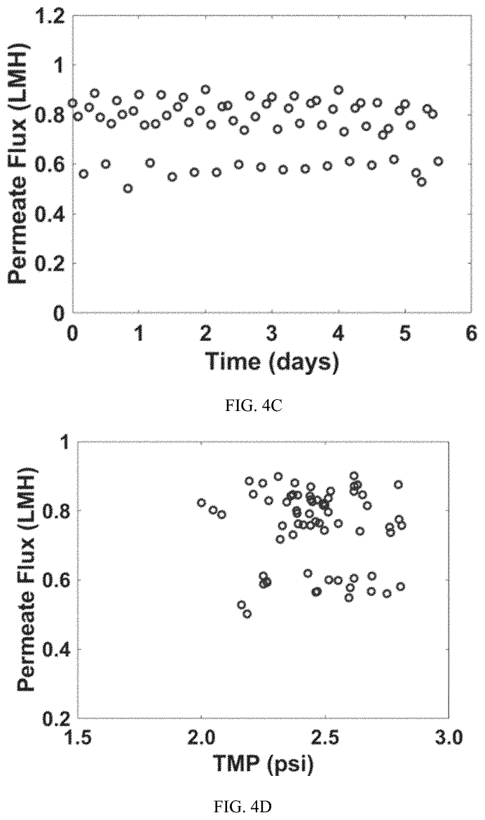

Data from the UF2 operation is shown in FIGS. 4A-4D. As shown in FIG. 4A, the recombinant therapeutic protein concentration remains relatively constant over time at around 175 g/L. In FIG. 4B, consistent flux/TMP values indicated filter integrity and absence of fouling throughout the course of operation. Throughout the course of operation, the permeate flux remained consistent (FIG. 4C). FIG. 4D showed that when the permeate flux was elevated, the transmembrane pressure was also elevated. Taken together, the data in FIGS. 4A-4D demonstrated that the bioprocessing system regulated and maintained the recombinant therapeutic protein concentration (at a higher level than UF1 or DF) and permeate flux during the UF2 operation.

As described above, the combined use of a back pressure regulator and a booster pump was demonstrated to facilitate a) single-pass continuous ultrafiltration and diafiltration of a recombinant therapeutic protein solution, b) integration of multiple UF and/or DF operations and filter configurations (series and/or parallel) with other purification processes, and c) the robust, independent, and integrated control for each UF and DF step. As shown for the DF operation, the combination of the back pressure regulator and the booster pump is required to maintain a state of control. Implementation of both feedback and feed-forward control provides the ability to maintain consistent retentate concentration in UF/DF, retentate flow rate, and achieve the desired diavolumes in continuous diafiltration.

The combined use of a back pressure regulator and booster pump described here has applications beyond TFF. By decoupling the pressure at the outlet of a unit operation from the pressure downstream of that unit operation, this method allows for the integration of unit operations in a continuous processing train without the need for intermediate break tanks or cascading pressure throughout the system. This method can be applied between every unit operation in a traditional bioprocessing system. These key features promote the translation of current biomanufacturing processes to an end-to-end integrated and continuous framework.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of processing a fluid comprising a recombinant therapeutic protein, the method comprising:
(a) providing a bioprocessing system comprising: (i) a unit operation, (ii) at least one inlet on the unit operation, (iii) at least one outlet on the unit operation, (iv) an inlet conduit in fluid communication with at least one inlet of the unit operation, (v) an outlet conduit in fluid communication with at least one outlet of the unit operation, (vi) a back pressure regulator disposed in the outlet conduit, and (vii) a booster pump disposed in the outlet conduit that follows downstream of the back pressure regulator;
(b) flowing the fluid comprising the recombinant therapeutic protein through the inlet conduit and into the at least one inlet of the unit operation, processing the fluid within the unit operation to obtain a processed fluid, and then flowing a portion of the processed fluid into the outlet conduit; and
(c) maintaining a target flow rate and/or pressure through the outlet conduit using the back pressure regulator and the booster pump.

2. The method of claim 1, wherein the unit operation is selected from the group consisting of a cell removal unit operation, a cell retention unit operation, a tangential flow filtration unit operation, a chromatography unit operation, a viral inactivation unit operation, a virus removal filtration unit operation, and a formulation unit operation.

3. A method of processing a fluid comprising a recombinant therapeutic protein, the method comprising:
(a) providing a bioprocessing system comprising: (i) a tangential flow filtration (TFF) unit with a permeate side and a retentate side, (ii) at least one inlet on the TFF unit, (iii) at least two outlets on the TFF unit, where one of the at least two outlets is on the permeate side of the TFF unit and one of the at least two outlets is on the retentate side of the TFF unit, (iv) an inlet conduit in fluid communication with at least one inlet of the TFF unit, (v) a permeate conduit in fluid communication with the outlet on the permeate side of the TFF unit, (vi) a retentate conduit in fluid communication with the outlet on the retentate side of the TFF unit, (vii) a back pressure regulator disposed in the permeate conduit or the retentate conduit, and (viii) a booster pump disposed in the permeate conduit or the retentate conduit that follows downstream of the back pressure regulator;
(b) flowing the fluid comprising the recombinant therapeutic protein through the inlet conduit and into the at least one inlet of the TFF unit, and separating the fluid into retentate and permeate streams based on the pore size or molecular weight cutoff of the TFF unit, and then flowing those permeate and retentate streams into the permeate and retentate conduits, respectively; and
(c) maintaining a target flow rate and/or pressure through the permeate conduit or the retentate conduit using the back pressure regulator and the booster pump.

4.-8. (canceled)

9. The method of claim 3, wherein the recombinant therapeutic protein in the fluid in the inlet conduit has a concentration from about 0.1 g/L to about 300 g/L.

10. (canceled)

11. The method of claim 3, wherein the recombinant therapeutic protein in the retentate stream in the retentate conduit has a concentration from about 0.1 g/L to about 300 g/L.

12. (canceled)

13. The method of claim 3, wherein the recombinant therapeutic protein in the permeate stream in the permeate conduit has a concentration from about 0 g/L to about 100 g/L.

14. (canceled)

15. The method of claim 3, wherein the TFF unit is comprised of one or more filters each with filter areas of about 50 cm² to about m².

16. (canceled)

17. The method of claim 1, wherein the booster pump is a dynamic pump.

18. The method of claim 17, wherein the dynamic pump is a centrifugal pump.

19. The method of claim 3, wherein step (b) comprises continuously flowing the fluid at an average rate from about 0.1 mL/minute to about 10 L/minute.

20. (canceled)

21. The method of claim 3, wherein the bioprocessing system further comprises one or more sensor(s).

22. The method of claim 21, wherein at least one of the one or more sensor(s) is disposed in the inlet conduit, the permeate conduit, and/or the retentate conduit.

23.-24. (canceled)

25. The method of claim 21, wherein the recombinant therapeutic protein concentration is measured using the one or more sensor(s) that has a sampling time of less than about 2 minutes.

26. The method of claim 3, wherein the recombinant therapeutic protein concentration is measured using UV absorbance, refractive index, Fourier Transform Infrared Spectroscopy (FTIR), or Raman spectroscopy.

27.-28. (canceled)

29. The method of claim 26, wherein the UV absorbance is measured with a flowcell with a fixed pathlength or a variable pathlength.

30. (canceled)

31. The method of claim 1, further comprises, prior to step (a), performing one or more second unit operation(s) on the recombinant therapeutic protein, wherein the one or more second unit operation(s) is/are integrated upstream of the bioprocessing system, such that one of the one or more second unit operation(s) is in fluid communication with the inlet conduit of the bioprocessing system.

32. The method of claim 31, wherein the one or more second unit operation(s) is/are selected from the group consisting of: a bioreactor unit operation, a cell removal unit operation, a cell retention unit operation, a chromatography unit operation, a viral inactivation unit operation, a virus removal unit operation, an ultrafiltration unit operation, and a diafiltration unit operation.

33. The method of claim 3, further comprises, after step (c), performing one or more second unit operation(s), such that one of the one or more second unit operation(s) is in fluid communication with the permeate conduit or the retentate conduit of the bioprocessing system.

34.-35. (canceled)

36. A bioprocessing system comprising:
(i) a unit operation;
(ii) at least one inlet on the unit operation;
(iii) at least one outlet on the unit operation;
(iv) an inlet conduit in fluid communication with at least one inlet of the unit operation;
(v) an outlet conduit in fluid communication with at least one outlet of the unit operation;
(vi) a back pressure regulator disposed in the outlet conduit, and
(vii) a booster pump disposed in the outlet conduit that follows downstream of the back pressure regulator,
wherein:
the bioprocessing system is configured to flow fluid comprising a recombinant therapeutic protein through the inlet conduit and into the at least one inlet of the unit operation, processing the fluid within the unit operation to obtain a processed fluid, and then flowing a portion of the processed fluid into the outlet conduit and
the back pressure regulator and the booster pump are configured to maintain a target flow rate and/or pressure through the outlet conduit.

37. (canceled)

38. A bioprocessing system.
comprising:
(i) a tangential flow filtration (TFF) unit having at least one inlet and at least two outlets, wherein one of the at least two outlets is on the permeate side of the TFF unit and one of the at least two outlets is on the retentate side of the TFF unit;
(ii) an inlet conduit in fluid communication with the at least one inlet of the TFF unit;
(iii) a permeate conduit in fluid communication with the outlet on the permeate side of the TFF unit;
(iv) a retentate conduit in fluid communication with the outlet on the retentate side of the TFF unit;
(v) a back pressure regulator disposed in the permeate conduit or the retentate conduit, and
(vi) a booster pump disposed in the permeate conduit or the retentate conduit that follows downstream of the back pressure regulator,
wherein:
the bioprocessing system is configured to flow fluid comprising a recombinant therapeutic protein through the inlet conduit and into the at least one inlet of the TFF unit, and separate the fluid into retentate and permeate streams based on the pore size or molecular weight cutoff of the TFF unit, and then flow those permeate and retentate streams into the permeate and retentate conduits; and
the back pressure regulator and the booster pump are configured to maintain a target flow rate and/or pressure through the permeate conduit or the retentate conduit.

39.-68. (canceled)
